(19) United States
(12) Patent Application Publication
(10) Pub. No.: US 2008/0153761 A1
(43) Pub. Date: Jun. 26, 2008

(54) Compositions for protection against superficial vasodilator flush syndrome, and methods of use

(76) Inventor: Theoharis C. Theoharides, Brookline, MA (US)

Correspondence Address: Dr. Melvin Blecher, 4329 Van Ness St., NW, Washington, DC, 20016-5625, US

(21) Appl. No.: 11/999,991

(22) Filed: Dec. 10, 2007

Related U.S. Application Data
(63) Continuation-in-part of application No. 11/651,161, filed on Jan. 10, 2007.
Application No. 11/651,161 is a continuation-in-part of application No. 10/811,823, filed on Mar. 30, 2004, now abandoned.
Application No. 10/811,823 is a continuation-in-part of application No. PCT/US02/00476, filed on Jan. 3, 2002.
Application No. PCT/US02/00476 is a division of application No. PCT/US02/00476, filed on Jan. 3, 2002.
Application No. PCT/US02/00476 is a continuation of application No. 09/771,669, filed on Jan. 30, 2001, now Pat. No. 6,984,667.
Application No. 09/771,669 is a continuation-in-part of application No. 09/056,707, filed on Apr. 8, 1998, now Pat. No. 6,689,748.
Application No. 09/056,707 is a division of application No. 09/056,707, filed on Apr. 8, 1998, now Pat. No. 6,689,748.

(51) Int. Cl. A61K 31/352; A61K 31/70; A61K 31/5415; A61K 31/517; A61K 31/445; A61K 31/4545; A61P 9/00

(52) U.S. Cl. 514/25; 514/456; 514/225.8; 514/266.21; 514/325; 514/318

(57) ABSTRACT

Compositions for protection against SVFS induced by niacin, a carcinoid, mesenteric fraction, serotonin, post-menopause, alcohol or monosodium glutamate, comprising a flavonoid compound of the structure 2-phenyl-4H-1-benzopyran or 2-phenyl-4-keto-1-benzopyran or glycosides thereof, administered alone or together with an anti-superficial vasodilation dose of one or more of a non-bovine sulfated proteoglycan, a D-hexosamine sulfate, a serotonin inhibitor, willow bark extract and an olive kernel extract. As much as 100% protection against niacin flush can be achieved by luteolin and quercetin alone. A composition for treating cardiovascular disease with niacin, but without eliciting the SVFS effects of niacin, has also been invented.

This application is a CIP of co-pending U.S. Ser. No. 11/651,161, filed Jan. 11, 2007 which was a CIP of co-pending U.S. Ser. No. 10/811,823, filed Mar. 30, 2004, which was a CIP/Div of co-pending PCT/US02/00476, filed Jan. 3, 2002 (now U.S. Pat. No. 1,365,777), which was a continuation of co-pending U.S. Ser. No. 09/771,669, filed Jan. 30, 2001 (now U.S. Pat. No. 6,984,667), which was a CIP/Div of co-pending U.S. Ser. No. 09/056,707, filed Apr. 8, 1998 (now U.S. Pat. No. 6,689,746.

### BACKGROUND OF THE INVENTION

The invention is generally related to the treatment of superficial vasodilator flush syndrome (“SVFS”). More specifically, the invention relates to compositions containing inhibitors of superficial vasodilators such as niacin, histamine, PGD2 and serotonin, for example, a flavonoid compound, alone or together with other inhibitors of superficial vasodilators such as a sulfated proteoglycan or a D-hexosamine sulfate, that are designed to be used as dietary supplements to conventional approved medications for protection against SVFS.

In spite of risk factors, better recognition and availability of more efficacious drugs for lowering serum cholesterol and triglycerides, mortality from cardiovascular disease continues to occur in ⅔ of patients treated with statins, and to increase worldwide by about 25% (Libby, P, Amer. Coll. Cardiol. 46:1225 (2005). Niacin (nicotinic acid) at 1-2 g/day, decreases LDL and triglycerides, while increasing HDL levels (Carlson, L A, J. Intern. Med. 258:94 (2005). Moreover, niacin and a statin together have superior lipoprotein lowering profile (Brown B G, et al., N. Eng. J. Med. 345:1583 (2001), as also shown for slow release niacin combined with lovastatin (Gupta E K et al., Heart. Dis. 4:124 (2002). However, a limiting adverse effect in patients receiving immediate or sustained release niacin is the rapid development of significant cutaneous warmth and itching, especially on the face. referred to as “flush,” that severely limits compliance (Gupta et al., supra).

Niacin-induced flush is thought to involve the release of prostaglandin D₂ (PGD₂) from the skin (Morrow J D et al., J. Invest. Dermatol. 98:812 (1992); Morrow J D et al., Prostaglandins 38:263 (1989), especially from macrophages (Meyers C D et al., Atherosclerosis (2006); Urade Y. et al., J. Immunol. 50:191 (1989). However, co-administration of acetylsalicylic acid (ASA) to reduce PGD₂ levels has not been particularly effective in blocking niacin flush (Dunn R T et al., J. Therap. 2:478 (1995); Cefali E A et al., Int. J. Clin. Pharmacol. Ther. 45:78 (2007). Consequently, molecules other than PGD₂ may be involved, such as histamine, vasoactive intestinal peptide (VIP) and vascular endothelial growth factor (VEGF) (Grutzkau A. et al., Mol. Cell. Biol. 9:875 (1998); Boesiger J. et al., J. Exp. Med. 188:1135 (1998), as well as serotonin released from platelets, enterochromaffin cells (Boushey R P et al., Curr. Treat. Opt. 49:355 (2002), and mast cells (Kushnir-Sukhov N M et al., J. Allerg. Clin. Immunol 119:498 (2006). Serotonin is a prime candidate because it is known to be involved in the flush associated with carcinoid syndrome (Boushey 2002, supra).

SVFS is not limited to niacin-induced flush. This syndrome is present in another group of human conditions that includes carcinoid-induced, mesenteric fraction induced, serotonin-induced, postmenopause-induced, alcohol-induced and monosodium glutamate-induced SVFS.

An important need, therefore, exists for compositions for administration to humans suffering from SVFS produced by a variety of etiologies. This need is particularly urgent in patients suffering from niacin-induced SVFS, particularly those suffering with coronary artery disease who must reduce serum triglycerides and LDL cholesterol, and who cannot tolerate niacin alone or together with statins. Such formulations have now been discovered, and are described below.

### SUMMARY OF THE INVENTION

The invention comprises anti-SVFS compositions for human use that consist of a flavonoid compound having the basic structure of 2-phenyl-4H-1-benzopyran or its 4-keto counterpart or their glycosides, used either alone or together with one or more of a group consisting of superficial vasodilation inhibitors sulfated proteoglycan, olive kernel extract (“OKE”), sulfated glucosamine, a serotonin inhibitor, willow bark extract, S-adenosylmethionine (“SAM”), histamine-1 receptor antagonists, histamine-3 receptor agonists, antagonists of the actions of corticotropin releasing hormone (“CRH”), caffeine, folic acid, polyunsaturated fatty acids, and polyamines, together with appropriate excipients and carriers, said compositions having improved anti-SVFS effects synergistic with each other and synergistic with available conventional clinical treatment modalities.

In preferred embodiments the flavonoid compound is quercetin, luteolin, genistein, myricetin and/or their respective glycosides.

Where a sulfated hexosamine is also present along with the flavonoid compound, the preferred sugar is D-glucosamine sulfate.

Where a sulfated proteoglycan is also present along with the flavonoid compound, the preferred composition is non-bovine chondroitin sulfate.

Where serotonin inhibitors are also used in anti-flush formulations, preferred compounds are prochlorperazine, cyproheptadine, azatadine and ketanserin.

In yet another embodiment, inventive compositions that protect humans against a variety of SVDS entities include a flavonoid compound alone or in combination with one or more of OKE, willow bark extract, a serotonin inhibitor a PGD2 inhbiitor, and a CRH inhibitor.

The novel OKE may be used to increase the absorption of difficulty-absorbable drugs across the intestine, skin, oral and nasal pulmonary alveoli.

In a preferred embodiment of the invention, a composition for treating cardiovascular disease has been devised that includes niacin but does not elicit the SVFS that normally accompanies administration of this drug.

### FIGURES

FIG. 1. shows the basic structure of flavonoid compounds, 2-phenyl-4H-1-benzopyran and its 4-keto counterpart, that are active in carrying out the invention. The active flavonoids active in the present invention differ only in the presence or absence of hydroxyl groups at ring positions 5, 7, 2′, 3′, 4′ and 5′.

FIG. 2. (A) A time-course of ear temperature increase (n=5) in response to a single intraperitoneal niacin (7.5 mg/rat) injection. All time points were significant (p=0.0002). (B) Dose-response of the effect of a single ip niacin injection on ear temperature increases recorded 45 min later (n=5). Niacin rat doses were based on 80 kg human (H) doses as follows: 5.0 mg/rat=1,167 mg/H, 7.5 mg/rat=1,750 mg/H; 10 mg/rat=2,334 mg/H (p=0.0001).

FIG. 3. Comparison of the inhibitory effect of fisetin, kaempferol, luteolin, myricetin, quercetin (4.3 mg/rat=1000 mg/80 kg), and ASA (1.22 mg/rat), administered ip 10 min prior to niacin on the ear temperature increase recorded 45 min after a single intraperitoneal injection of niacin (7.5 mg/rat) in olive oil (n=6,*p=0.0204, **p=0.0041, ***p=0.0002, ****p=0.0193). The percent inhibition was calculated after the corresponding baseline temperature was subtracted.

FIG. 4. Time course of the inhibitory effect of luteolin 4.3 mg/rat pre-treatment (0 luteolin added together with niacin) on ear temperature increase (n=3) in response to a single ip injection of niacin (7.5 mg/rat) measured 45 min later. All time points were statistically significant as compared to a control rat injected with 0.5 ml olive oil and 7.5 mg niacin. Brackets indicate groups compared (*p <0.001).

FIG. 5. Effect of acetylsalicylic acid (“ASA”) (1.22 mg/rat) and luteolin (4.3 mg/rat) administered 2 hr prior to a single ip injection of niacin (7.5 mg/rat) on plasma PGD₂ levels measured 45 min later (n=3). Bracket indicate groups compared (*p=0.014; **p=0.0419).

FIG. 6. Effect of ASA (1.22 mg/rat) and luteolin (4.3 mg/rat) administered 2 hr prior to a single ip injection of niacin (7.5 mg/rat) on plasma serotonin levels measured 45 min later (n=3). Brackets indicate groups compared (*p=0.0263).

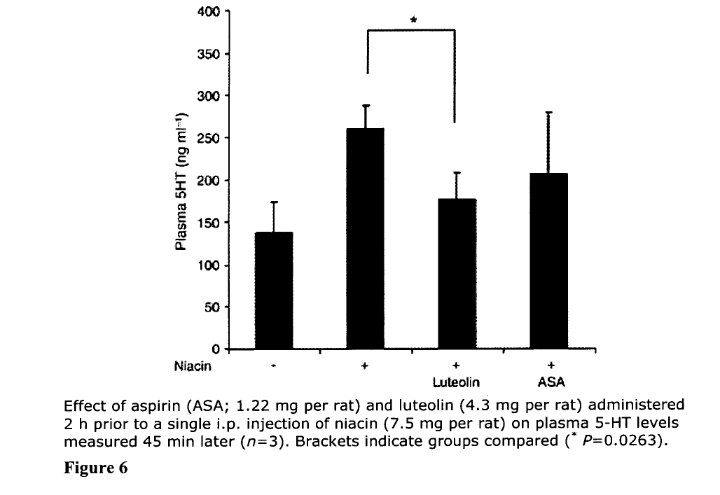

### DETAILED DESCRIPTION OF THE PREFERRED EMBODIMENTS

It has been discovered that a flavonoid compound or its glycoside counterpart, either alone or in combination with one or more of a group of vasodilation inhibitors consisting of a sulfated proteoglycan, OKE, a sulfated D-hexosamine, a serotonin inhibitor, a willow bark extract, a CRH inhibitor, a histamine-1 receptor antagonist, a histamine-3 receptor agonist, a polyamine, rutin and caffeine, have synergistic anti-SVFS effects, where SVFS is caused by to ingestion of niacin or to other SVFS inducers listed supra. OKE may be used to improve the transmembrane transport of difficulty-absorbable drugs in the intestine, skin, nasal, oral and pulmonary alveoli.

The preferred flavonoid compounds are luteolin and quercetin. In addition, other flavonoid compounds suitable in carrying out the invention include the quercetin glycoside rutin, myricetin, kaempferol glycoside astragaline. genistein, kaempferol, and the isoflavone phenoxodiol.

The preferred sulfated proteoglycan is non-bovine chondroitin sulfate. Other natural sulfated proteoglycans suitable for practicing this invention include keratan sulfate, dermatan sulfate and hyaluronic acid sodium salt (sodium hyaluronate). The preferred biological source of the chondroitin sulfate is shark cartilage because this source is more-highly sulfated than the common commercial chondroitin sulfate isolated from cow trachea. The shark cartilage source also avoids the potential dangers associated with bovine sources, such as bovine encephalopathy (“mad cow disease”).

The preferred D-hexosamine sulfate is D-glucosamine sulfate.

The OKE component of the inventive compositions is, referably, an unrefined (first pressing, filtered, oleic acid-related acidity <3%, water content <1%) extract product produced, for one source, on the island of Crete in Greece. This kernel extract product is especially prepared by applicant's process consisting essentially of: (1) harvesting first collection ripe olives, preferably in December; (2) compressing the oil from the flesh of the ripe olives; (3) washing the kernels remaining after step (2) with water to remove debris; (4) drying the washed kernels with a stream of hot air; (5) crushing the dried kernels to produce an extract; (6) extracting the extract from step (5) with an organic solvent (e.g., hexane, heptane, octane) plus steam; (7) removing particulate matter from the organic extract by centrifugation or microfiltering through 1-2 micron pore size filters; (8) evaporating the organic solvent and water from the clarified extract of step (7) by maintaining the extract at 86-100° C. while percolating helium (to avoid oxidation) through the fluid, which process reduces the water content to <1%, the acidity (as oleic acid) to <3%; and, the organic solvent to <1%; and (8) storing the final kernel extract product in the absence of air.

The inventive OKE surprisingly has the unique property of increasing absorption of the other components of the anti-inflammatory compositions through the intestinal mucosa or skin, and also adds its own content of important anti-oxidants, such as omega fatty acids (e.g., eicosapentanoic acid) and alpha tocopherol. The polyphenols found in such olive kernel extracts also have anti-inflammatory effects in, for example, arthritis [Martinez-Dominguez et al., Inflamm. Res. 50:102 (2001)]. E.B.E.K., Inc., Commercial, Industrial Enterprises of Crete, 118 Ethnikis Antistasecos, Heraklion, Crete, 71306, Greece, or MINERVA Edible Oils, 165 Tatoiou St., Athens, 14452, Greece, will prepare the extract product according to applicant's above-described procedure for commercial users. Parallel experiments with codfish oil, corn oil and olive oil (from the flesh of the olive) were comtemplated, but chondroitin sulfate solubility in these oils was insufficient to meet the requirements of the experiment.

In addition to its usefulness in increasing the absorption of the inventive compositions across the intestinal wall and the skin, the inventive OKE product is useful in aiding the dissolution of other drugs prior to administration to a patient, and is useful in promoting the absorption of other difficulty-absorbable drugs across intestinal mucosa, oral mucosl, nasal mucosa, and skin of patients.

In experiments with rat models of the SVFS, to be described in detail infra, applicant has surprisingly also discovered that serotonin mediates the flush syndrome induced by niacin administration. This discovery has opened up a new therapeutic approach for niacin flush. Applicant has discovered that serotonin inhibitors/antagonists such as prochlorperazine, cyproheptadine, azatadine and ketanserin, when used alone or in combination with the basic composition of the invention, inhibit the niacin flush syndrome

Another optional supplement to the basic compositions of the invention is a histamine-1 receptor antagonist, such as hydroxyzine, mezelastine, azelastine, azatadine, rupatadine and cyproheptadine. Other histamine-1 receptor antagonists are described in Table 25-1 in Goodman and Gilman's The Pharmaceutical Basis of Therapeutics, 9^{th} ed., New York, 1996. Histamine-3 receptor agonists are described in the Theoharides patents listed above.

The preferred concentration range of the proteoglycan, hexosamine sulfate and flavonoid components of the oral formulations are 50-3,000 mg per tablet or capsule. Generally, where present, the amounts of OKE are equal to those of the other active ingredients, preferably 50-1500 mg. The number of capsules or tablets to be taken per day is determined by the nature and severity of the medical condition, and is readily determinable by the patient's health provider. Other representative formulations are described in the examples infra.

The compositions of the invention may be formulated in any standard means of introducing pharmaceuticals into a patient, e.g., by means of tablets or capsules. Standard excipients and carriers for the active ingredients of the inventive compositions are described in Remington's Pharmaceutical Sciences, Mack Publishing Co., Easton, Pa.

### EXAMPLES

### Example 1

### Treating Niacin-Flush in Humans

Four normal male subjects (29±3 years) were entered in the following protocol: On days 1 and 2, they were administered 1 gm immediate release niacin, at 2 pm. On days 3 and 4 they were administered 2 capsules of a composition containing 150 mg quercetin and 450 mg of OKE per capsule. On days 4 and 6, they were administered two capsules at 8 am and 1 g niacin at 2 pm. Skin temperature was measured with an infrared digital pyrometer at 4 facial sites (forehead, both checks and chin) at 15, 30, 45, 60, 75 and 90 min post niacin administration, along with daily room temperature subjects also completed a symptoms questionnaire (erythema, edema, pruritus and burning sensation) on a scale of 0=no symptoms and 5=maximal symptoms. There was no significant increase in temperature rise with niacin administration, but symptoms (especially erythema and burning) ranged 4-5 and lasted 3-4 hrs. After administration of the inventive composition, the scores were reduced to 2-3 and lasted only about 75 min. (>50% inhibition). These results demonstrate that the inventive compositions containing a flavonoid will reduce niacin flush.

| | | | Niacin + | |
|---|---|---|---|---|
| | Niacinalone | | InventiveComposition | |
| Erythema | 4.75 ± 0.5 | 4.5 ± 0.58 | 3.25 ± 0.5 | 2.5 ± 0.58 |
| Edema | 0.5 ± 0.58 | 0.5 ± 0.58 | 0.25 ± 0.5 | 0.25 ± 0.5 |
| Urticaria | 2.25 ± 0.5 | 2.0 ± 0.82 | 1.75 ± 0.5 | 1.25 ± 0.5 |
| Burning | 4.75 ± 0.5 | 4.0 ± 0.82 | 3.0 ± 0.82 | 2.5 ± 0.58 |
| Duration (hr) | 3.63 ± 1.11 | 2.75 ± 0.87 | 1.68 ± 0.40 | 1.68 ± 0.70 |

### Example 2

### Protection Against Niacin Flush in an Animal Model

### Materials and Methods

Male Sprague-Dawley rats (300-350 g) were housed three per cage and were provided with food and water ad libitum. The room temperature was kept constant at 21±1° C., with a 14:10 hour light:dark schedule and lights out at 19:00 hour. ASA, fisetin, kaempferol, luteolin, myricetin, niacin, and quercetin were purchased from Sigma (St. Louis, Mo.). All drugs were first dissolved in OKE and then 0.9% NaCl fresh each day of the experiment.

### Assessment of Niacin-Induced Skin Temperature Changes

Temperature measurements were recorded with a hand-held infrared pyrometer connected to a millivoltmeter (Model OS613A, Omega Co., Stamford, Conn.). The probe was held at a distance of 1-2 mm from the animal's skin and temperature readings were taken from an ear area approximately 3 mm in diameter. Animals were habituated to handling and to the infrared probe for 3 days before use. On the day of the experiment, the animals were brought into the lab (9-10 AM). Three temperature readings from the top half of each ear were recorded for each time point without anesthesia immediately before animals were injected intraperitoneally (ip) with either niacin or the test flavonoid. The ear temperature was then measured every 10 min for a period up to 60 min. The animals were returned to their cages between measurements. Animals were “rested” for one week and were used again; the effect of niacin was not changed in rats that were used more than once.

Pre-Treatment with Various Flavonoids

Rats were randomly administered either (A) vehicle (olive kernel extract) followed by niacin or (B) a flavonoid (4.3 mg/rat, equivalent to 1,000 mg/80 kg human) followed by niacin. This dose of flavonoids, the structures of which differs only by 1 hydroxyl group at certain positions, was chosen because it was previously shown to be attainable in vivo (Kimata et al., 2000a) by oral administration.

### Blood Mediator Measurements

In certain cases, blood was collected immediately after the end of the experimental period by sacrificing the animal wish asphyxiation over CO₂ vapor decapitation and collection from neck vessels. Blood was centrifuged at 350×g in a refrigerated centrifuge, the plasma collected and frozen at −20° C. until assay. Plasma levels of PGD₂ (Cayman) and serotonin (Biosource, Belgium) were assayed by ELISA kit (Biosource, Belgium). The lowest levels of sensitivity for each were 200 pg/ml (intra and inter-assay variation 10-20%) and 0.5 ng/ml (intra-assay variation 26 and inter-assay variation 15), respectively.

### Statistical Analysis

The six ear temperature measurements (three from each ear) were averaged for each point. Any temperature change was calculated by subtracting from the mean value for each experimental point the baseline temperature obtained immediately before the vehicle/drug was injected or the baseline measured immediately before niacin administration, whichever was appropriate. All data are presented as mean ±SD of the actual temperatures or percent change from that recorded after niacin administration. Paired comparisons between niacin and control or niacin and drug pretreatment followed by niacin were analyzed with either the paired t-test or the non-parametric Mann-Whitney U test. Multi-variant ANOVA analysis was performed on all other comparisons. Significance is denoted by p<0.05.

Niacin was administered to unanesthetized rats, using 3 animals per dose.

### A: Effect of Niacin on Skin Temperature

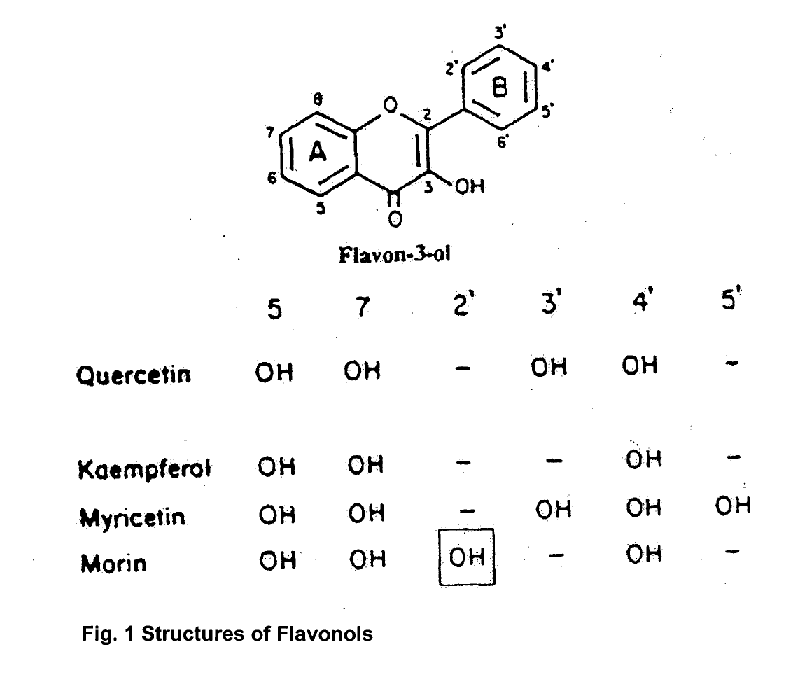

The basal mean ear temperature was 26.5-28.5 C (n=27). Niacin (7.5 mg/rat, equivalent to 1,750 mg/80 kg human) administered ip in conscious rats induced a time-dependent temperature increase with a maximum 1.9±0.2° C. (n=5, p=0.0002) at 45 min (FIG. 1A). A dose-response of niacin (5-10 mg/rat, n=5) showed maximal temperature increase of 2.0±0.1° C. (p=0.001) at 45 min with 7.5 mg/rat (FIG. 1B).

B: Treatment with Azatadine (Histamine-1 Receptor Antagonist) and Serotonin Receptor Antagonist.

Rats were treated with 1 ug of azatadine i.p. at time zero. Niacin, 5 mg, was given i.p. 45 mins. mins. post-azatadine, and ear temperatures were measured.

At 10 mins., azatadine had reduced the niacin +2 degrees C. effect by 75%.

C: Treatment with Cyproheptadine (Strong Histamine H1 and Serotonin Receptor Antagonist)

Rats were treated with 8.55 ug of the antagonist i.p. at time zero, and niacin, 5 mg, was given at 120-480 mins. thereafter. Ear temperatures were measured at 45 mins. after niacin.

There was no effect of niacin in animals pre-treated with cyproheptadine (100% inhibition).

D: Treatment with Ketotifen (Histamine-1-Receptor Antagonist)

Rats were pretreated with 17.1 ug of ketotifen, and niacin, 5 mg, was administered i.p. 30 mins. thereafter. Ear temperatures were measured 45 mins. after niacin.

The drug had no significant effect on the niacin effect.

E: Treatment with Quercetin.

Quercetin, 4.7 mg, was given to rats i.p. at time zero, and 5 ng niacin administered i.p. 120, 240 and 360 mins. thereafter.

### Quercetin Inhibited the Niacin Effect by 100%.

F: Effect of ASA and flavonoids on niacin-induced skin temperature increase

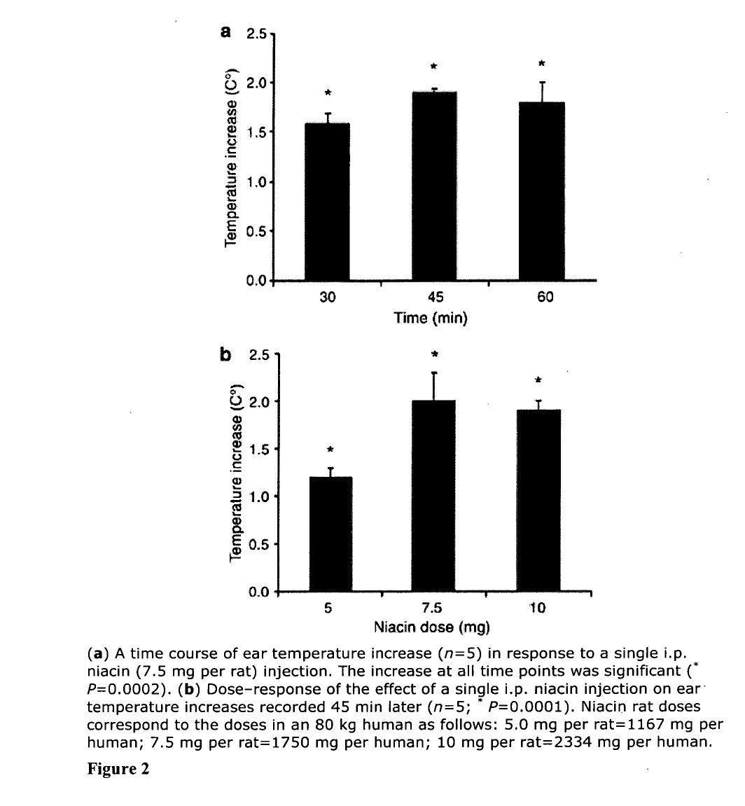

We investigated whether pretreatment for 2 hr with ASA (1.22 mg/rat equivalent to 325 mg/80 kg human) or various flavonoids (4.3 mg/kg, equivalent to 1,000 mg/80 kg human) could inhibit niacin's effect (7.5 mg/rat) in this animal model. ASA inhibited this effect by 30% (n=6, p=0.0193, FIG. 2). Myricetin and kaempferol had no effect; fisetin inhibited the effect of niacin by 50% (n=6, p=0.0204, FIG. 2). Quercetin and luteolin were the most effective in reducing ear temperatures by 96% and 88%, respectively (n=6, p=0.0002 and p=0.0041, FIG. 2); there was no statistical difference between the effects of quercetin and luteolin.

G: Effect of Pretreatment Duration with Luteolin

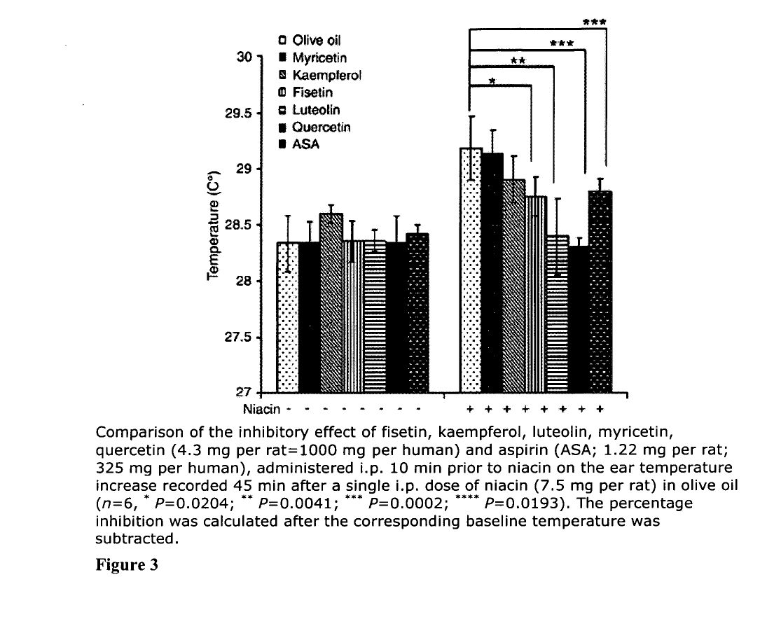

We then investigated whether the length of pretreatment with luteolin affected its ability to inhibit the niacin flush. Luteolin significantly decreased the niacin induced temperature increase even when added together with niacin (time=0) and remained significant at all time points from 0 to 6 hours. There was no significant difference between the 2, 4, and 6 hour pretreatment time points amongst the luteolin pretreated samples (FIG. 3).

### H: Effect of Niacin on Plasma PGD₂ and Serotonin Levels

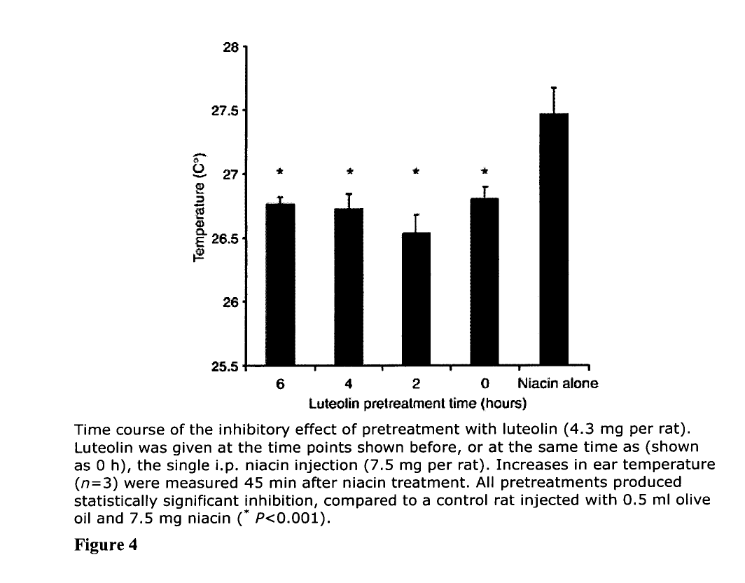

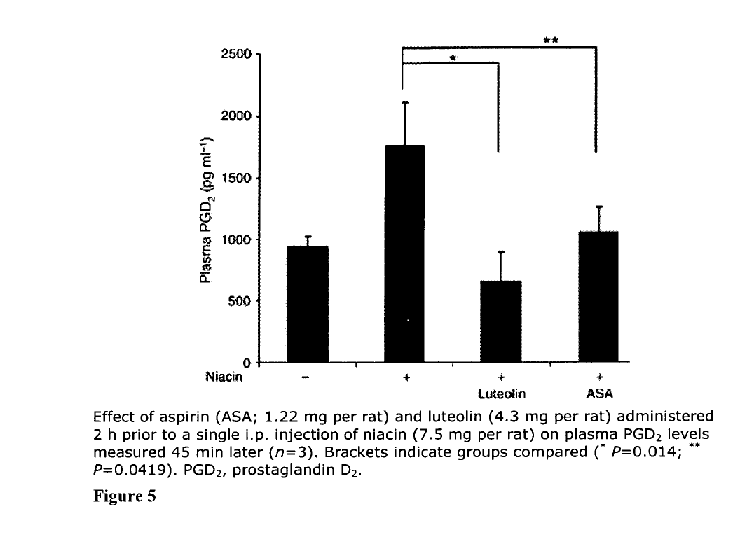

We then investigated the effect of niacin, as well as the effect of the luteolin, on niacin-induced plasma PGD₂ and serotonin levels. Niacin (7.5 mg/rat) increased plasma PGD₂ by 88% from 933±94 pg/ml to 1750±352 pg/ml at 45 min (n=3, p=0.0178, FIG. 4) and plasma serotonin by 90% from 137±37 ng/ml (n=4) to 260±28 ng/ml (n=4, p=0.0101, FIG. 5).

### I: Effect of ASA and Luteolin on Niacin-Induced Plasma PGD₂ and Serotonin Levels

Pretreatment for 2 hr with ASA (1.22 mg/rat) reduced plasma PGD₂ by 86% (n=3, p=0.018, FIG. 4), but had no statistically significant inhibitory effect on plasma serotonin levels (FIG. 5). In contrast, luteolin (4.3 mg/rat) significantly reduced plasma PGD₂ levels by 100% (n=3, p=0.014, FIG. 4), and serotonin levels by 32% (n=3, p=0.0263, FIG. 5). If the baseline serotonin of 137 ng/ml were to be subtracted from the niacin-induced level of 260 ng/ml and the level of 177 ng/ml in the presence of both luteolin and niacin, and then calculate the inhibition, it now becomes 97%.

### Example 3

### A Representative Example of a Composition for Protecting Against SVFS

| Ingredients, | per capsule: | |
|---|---|---|
| Luteolin | 250 | mg |

Optionally:

| | | |
|---|---|---|
| Chondroitin sulfate | 50 | mg |
| D-glucosamine sulfate | 90 | mg |
| Olive kernel extract | 450 | mg |
| Willow bark extract | 100 | mg |
| Cyproheptadine or | 4 | mg |
| azatadine | | |

### Example 4

### A Representative Composition for Treating Cardiovascular Defects That Contains Niacin But Does Not Exhibit SVFS

| | Amount per 2 #120 Softgel Capsules* | |
|---|---|---|
| Luteolin | 300 | mg |
| Niacin | 300 | mg |
| S-adenosylmethionine | 200 | mg |
| Folic acid | 140 | ug |
| Eicosapentenoic Acid or | 100-200 | mg |
| Docosahexenoic acid | | |

Optionally:

| | | |
|---|---|---|
| OKE | 50-1500 | mg |
| Willow bark extract | 200 | mg |

*The number of capsules to be taken per day will depend on the status of the cardiovascular condition in the patient.

### Example 5

### Effect of Olive Kernel Extract on Absorption of a Proteoglycan Sulfate

### In Vivo

Chondroitin sulfate (Type C from shark cartilage) was tritiated by New England Nuclear Corp. to a specific activity of 4.3 mCi/ml.

Unlabeled chondroitin sulfate was dissolved in OKE at a ratio of about 55 w/v chondroitin sulfate powder to about 450 w/v of OKE (2.9% acidity as oleic acid, 1.03% water, 0.08% hexane). To this solution was added 20.2 microcuries of the labeled chondroitin sulfate. AAA gelatin capsules were filled with the resulting solution using an aluminum template molding device.

The laboratory animals (250 g male Sprague-Dawley rats) were kept overnight without food but with free access to water. One capsule containing the above-described chondroitin sulfate-OKE solution was given to each rat per os. Control animals were given the equivalent amount of chondroitin, but without olive kernel extract. The animals were then given free access to food. Serum radioactivity was measured 8 hours thereafter in a beta scintillation counter.

The results showed that, in control animals, about 3.9%+/−0.4% (n=3) of the dose of labeled chondroitin sulfate reached the circulation. In sharp contrast, in animals given the OKE along with the labeled chondroitin sulfate, about 14.3%+/−0.7% (n=4) of the dose was absorbed into the general circulation.

These results demonstrate that OKE increased by almost 400% the absorption of a proteoglycan from the intestine into the general circulation.

I claim:

1. A composition, said composition comprising a flavonoid compound of basic structure 2-phenyl-4H-1-benzopyran or 2-phenyl-4-keto-1-benzyopyran, or a glycoside thereof, wherein said composition exhibits the property of protecting humans against superficial vasodilator flush syndrome (“SVFS”) induced by administered niacin.

2. The composition of claim 1, wherein said flavonoid compound is selected from the structural/functional group consisting of quercetin, luteolin, myricetin and genistein, or a glycoside derivative of said flavonoids.

3. The composition of claim 2, wherein said flavonoid composition is supplemented with one or more additional anti-SVFS compounds.

4. The composition of claim 3, wherein said additional anti-SVFS compound is a heavily sulfated non-bovine proteoglycan.

5. The composition of claim 4, wherein said proteoglycan is chondroitin sulfate.

6. The composition of claim 3, wherein said additional anti-SVFS compound is a hexosamine sulfate.

7. The composition of claim 6, wherein said hexosamine sulfate is D-glucosamine sulfate. supplemented.

8. The composition of claim 3 wherein said supplemental anti-SVFS compound is a serotonin inhibitor.

9. The composition of claim 8, wherein said inhibitor is a serotonin receptor antagonist.

10. The composition of claim 9, wherein said antagonist is prochlorperazine or ketanserin.

11. The composition of claim 8, wherein said serotonin inhibitor is a mixed histamine-1 and serotonin receptor antagonist selected from the group consisting of cyproheptadine or azatadine.

12. A composition, said composition comprising a flavonoid compound of basic structure 2-phenyl-4H-1-benzopyran or 2-phenyl-4-keto-1-benzyopyran, or a glycoside thereof, wherein said composition exhibits the property of protecting humans against SVFS induced by a member of the group consisting of a carcinoid-associated flush, mesenteric fraction-induced flush, serotonin-induced flush, post-menopausal-induced flush, alcohol-induced flush and monosodium glutamate-induced flush.

13. The composition of claim 12, wherein said flavonoid compound is selected from the group consisting of quercetin, luteolin, myricetin and genistein, and a glycoside derivative of said flavonoids.

14. The composition of claim 13, wherein said flavonoid composition is supplemented with an additional anti-SVFS compound.

15. The composition of claim 14, wherein said anti-inflammatory compound is a heavily sulfated proteoglycan.

16. The composition of claim 15, wherein said proteoglycan is chondroitin sulfate.

17. The composition of claim 14, wherein said additional anti-SVFS compound is a hexosamine sulfate.

18. The composition of claim 17, wherein said hexosamine sulfate is D-glucosamine sulfate.

19. The composition of claim 14 wherein said additional anti-SVFS compound is a serotonin inhibitor.

20. The composition of claim 19, wherein said inhibitor is a serotonin receptor antagonist.

21. The composition of claim 20, wherein said antagonist is prochlorperazine or ketanserin.

22. The composition of claim 19, wherein said serotonin inhibitor is a mixed histamine-1 and serotonin receptor antagonist selected from the group consisting of cyproheptadine or azatadine.

23. A method for protecting an individual from the SVFS effects induced by niacin intake comprising administration to said individual effective doses for effective periods of time of any one or more of the compositions listed in claims 1 and 3.

24. A method for protecting an individual from the SVFS effects associated with carcinoid-associated flush, mesenteric fraction-induced flush, serotonin-induced flush, post-menopausal-induced flush, alcohol-induced flush and monosodium glutamate-induced flush. comprising administration to said individual effective doses for effective periods of time of any one or more of the compositions listed in claims 12 and 14.

25. A composition for treating a cardiovascular condition with niacin but without eliciting the SVFS effect of niacin, comprising niacin, luteolin, SAMe, folic acid, and a long-chain polyunsaturated fatty acid, and, optionally, olive kernel extract or willow bark extract or both, in amounts effective for treating said cardiovascular condition.

26. A method for treating a cardiovascular condition in a patient with niacin, but without eliciting the SVFS effect of said niacin, comprising the administration to said patient of clinically effective amounts of the composition of claim 25.
